(19) United States
(12) Patent Application Publication
(10) Pub. No.: US 2006/0005325 A1
(43) Pub. Date: Jan. 12, 2006

(54) Leave-in cosmetic composition comprising at least one elastomeric film-forming polymer and use thereof for conditioning keratin materials

(76) Inventors: Henri Samain, Bievres (FR); Isabelle Rollat-Corvol, Paris (FR); Jonathan Gawtrey, Boulogne (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/171,211

(22) Filed: Jul. 1, 2005

Related U.S. Application Data
(60) Provisional application No. 60/587,856, filed on Jul. 15, 2004.

(30) Foreign Application Priority Data
Jul. 1, 2004 (FR) 0407304

(51) Int. Cl. A61K 8/00

(52) U.S. Cl. 8/405; 8/406

(57) ABSTRACT

Disclosed is a leave-in cosmetic composition comprising, in a cosmetically acceptable medium, at least one elastomeric film-forming polymer chosen such that the film obtained by drying this polymer, in an aqueous or alcoholic medium, at ambient temperature and at a relative humidity of 55%, has a mechanical profile comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%.

This application claims benefit of U.S. Provisional Application No. 60/587,856, filed Jul. 15, 2004, the contents of which are incorporated herein by reference. This application also claims benefit of priority under 35 U.S.C. § 119 to French Patent Application No. 04 07304, filed Jul. 1, 2004, the contents of which are also incorporated by reference.

Disclosed herein is a cosmetic composition comprising, in a cosmetically acceptable medium, at least one elastomeric film-forming polymer with specific characteristics. Also disclosed herein is the use of this composition for formulating leave-in products intended for human keratin materials such as the skin, the nails, and the keratin fibers. These products may be used as care products for the hair, the skin, or the eyelashes, for example, as hair protection and repair products or styling products. Further disclosed herein is a method for styling, for shaping, or for maintaining keratin fibers, for example, the hair, using this composition.

The setting of the hairstyle is an important element of styling, and involves maintaining a shape that has already been formed. As used herein, the term “styling composition” refers to any type of hair composition that can be used for carrying out the styling.

Examples of widely available hair products for setting the hair include compositions that are in aerosol or pump-action spray bottles, such as lacquers, sprays and mousses. These compositions typically comprise a solution, for example, alcoholic and aqueous-alcoholic solutions, and at least one film-forming polymer that is water-soluble or soluble in alcohol, mixed with various cosmetic adjuvants, or alternatively, products to be applied by hand, such as gels and waxes.

However, these hair compositions for styling, especially aerosol sprays and lacquers, do not always allow the hairstyle to satisfactorily withstand the various natural movements that occur in daily life, such as walking, head movements, and gusts of wind. In addition, these compositions give the hair a feeling of stiffness, called the “helmet effect”. Styling shampoos as styling products are also known.

Polymers conventionally used for formulating hair products for styling are anionic, amphoteric, or non-ionic film-forming polymers that may result in the formation of films having a relatively hard and brittle nature. When the polymer film is too brittle, the percentage elongation at break measured on the film may be low, i.e., generally less than 2%, with the result that the hairstyle does not hold over time.

To remedy this problem, these polymers may be mixed with plasticizers to obtain coatings that are more flexible and not friable. However, these films are deformable and plastic, i.e., after deformation, they may only very slightly recover their initial shape. Although the hold of the hairstyle is improved, it may not yet be satisfactory since the shape of the hairstyle may change over time.

It is therefore desirable to provide cosmetic compositions for maintaining and/or setting the hairstyle that provide the hair with, besides a long-lasting set, ease of use, and/or good cosmetic properties, such as conditioning the head of hair so as to confer successful untangling, softness, and/or a pleasant, non-tacky appearance.

Moreover, products having care, protective and/or conditioning properties are also sought for the skin, for example, human facial skin, and the integuments, for example, eyelashes and eyebrows.

In addition, whether for the hair or the skin, it is also desirable to provide cosmetic compositions capable of forming, on these keratin materials, a flexible film that follows the movements of the skin or the hair, without any effect of tautness or heaviness or a feeling of rigidity.

The present inventors have discovered that it is possible to remedy at least one of the technical problems mentioned above, using at least one specific elastomeric film-forming polymer in a leave-in composition.

One embodiment of the present disclosure, therefore, relates to a leave-in cosmetic composition comprising, in a cosmetically acceptable medium, at least one elastomeric film-forming polymer chosen such that the film obtained by drying the at least one elastomeric polymer at ambient temperature (22° C.±2° C.) and at a relative humidity of 55%±5%, has a mechanical profile comprising:
(a) an elongation at break (ε_{b}) of greater than or equal to 800%,
(b) an instantaneous recovery (Rᵢ) of at least 75%, after an elongation of 150%, and
(c) a recovery (R₃₀₀) at 300 seconds of greater than 80%.

This composition makes it possible to obtain a flexible film that is not brittle on human keratin materials and that follows their movement.

As used herein, the term “at least one” elastomeric film-forming polymer is intended to mean one or more (2, 3, or more) elastomeric film-forming polymers.

As used herein, the term “leave-in composition” is intended to mean any composition that is formulated so as to remain on the human keratin materials, such as the hair, the skin, and the eyelashes, after application.

The composition according to at least one embodiment of the present disclosure may provide human keratin materials, for example, the hair, with conditioning and/or care and also may provide hair with a very high level (highly set) styling effect and an excellent hold of the hairstyle over time. The composition may be a leave-in hair composition, for example, a conditioner (or conditioning composition) which may be applied after shampooing, dyeing, bleaching, relaxing, and/or permanent-waving of the hair.

The leave-in composition may be in any of the conventional forms of leave-in cosmetic compositions, for example, gels; lotions; creams; waxes; mousses; conditioners; conditioning agents; lotions for rinsing the hair, to be applied after or before the application of a composition for permanent-waving, waving, relaxing, and/or dyeing the hair; and body hygiene products such as care products for the body, the face, the neck, and the eyes, for example, serums, lotions, creams, gels, or milks.

In at least one embodiment, the composition may be in a form chosen from hair mousses, gels, lotions, and creams that may have a styling effect and may also have a conditioning effect. As used herein, the expression “conditioning effect on keratin materials” is intended to mean an effect chosen from untangling, smoothing out, gloss, and volume. In one embodiment, the composition may be in a form chosen from styling mousses and gels to be applied to dry or wet hair.

Also disclosed herein is a composition, in the form of a mousse or gel, for styling the hair, to be used without rinsing, comprising a cosmetically acceptable medium and at least one elastomeric film-forming polymer as defined above. This mousse or gel may be taken up with the fingers or sprayed onto the hair using a pump-action bottle.

Further disclosed herein is a method for shaping or maintaining the hairstyle, comprising the use of this composition.

Another embodiment of the present disclosure relates to a method for caring for and/or protecting the skin, including the scalp, and/or human keratin fibers, such as the hair, the eyelashes and the eyebrows, comprising applying to the skin and/or said human keratin fibers a cosmetic composition comprising, in a cosmetically acceptable medium, at least one elastomeric film-forming polymer as defined above. The skin care may include repairing the skin against the ravages of time, such as dryness, wrinkles and fine lines, and softness. In at least one embodiment, the composition may be applied to protect the skin against outside stimuli such as wind, pollution, and/or UV radiation.

A further embodiment of the present disclosure relates to a method for conditioning human keratin materials, such as the skin, including the scalp, and/or human keratin fibers, comprising applying to the keratin materials a cosmetic composition comprising, in a cosmetically acceptable medium, at least one elastomeric film-forming polymer as defined above.

Another embodiment of the present disclosure relates to a composition intended for the care and/or protection of human keratin materials such as the skin, the hair, the eyelashes, and the nails, comprising a cosmetic composition comprising, in a cosmetically acceptable medium, at least one elastomeric film-forming polymer as defined above. Yet another embodiment of the present invention relates to producing a cosmetic hair product for the purpose of maintaining and/or setting the hairstyle.

As used herein, the expression “film obtained by drying at ambient temperature (22° C.±2° C.) and at a relative humidity of 55%±5%” is intended to mean the film obtained, under these conditions, from a mixture containing 6% of active material (a.m.) of elastomeric film-forming polymer in a mixture of 30% by weight of ethanol and 70% by weight of water, relative to the total alcohol+water weight, the amount of mixture being adjusted so as to obtain, in a Teflon matrix, a film 500 μm±50 μm thick. The drying is continued until the weight of the film no longer changes, approximately 12 days. The elongation at break and the recovery rate are evaluated by means of the tests described below.

To carry out the tensile tests, the film is cut up into rectangle-shaped test pieces, 80 mm long and 15 mm wide.

The tests are carried out on a device sold under the name Lloyd or sold under the name Zwick, under the same temperature and humidity conditions as for the drying, i.e., a temperature of 22° C.±2° C. and a relative humidity of 50%±5%.

The test pieces are drawn at a speed of 20 mm/min and the distance between the jaws is 50±1 mm.

To determine the instantaneous recovery (Rᵢ), the following procedure is carried out:
the test piece is drawn by 150% (εₘₐₓ), i.e., 1.5 times its initial length (l₀),
the stress is released by applying a return speed equal to the tensile speed, i.e. 20 mm/min, and the percentage elongation of the test piece is measured, after a return to zero constraint (εᵢ).

The % instantaneous recovery (Rᵢ) is given by the formula below:
Rᵢ=((εₘₐₓ−εᵢ)/εₘₐₓ)×100

To determine the recovery at 300 seconds, the test piece, having been subjected to the above operations is maintained at zero stress for a further 300 seconds, and its percentage elongation (ε₃₀₀ₛ) is measured.

The % recovery at 300 seconds (R₃₀₀ₛ) is given by the formula below:
R₃₀₀ₛ=((εₘₐₓ−ε₃₀₀ₛ)/εₘₐₓ)×100

In at least one embodiment, the at least one polymer disclosed herein, optionally combined with a plasticizer and/or a film-forming agent, is such that the polymer(s) form, under the conditions of the tests above, a film having an elongation at break ranging from 800% to 3000%; having an instantaneous recovery of 75% to 100%; and a recovery at 300 seconds ranging from 85% to 100%.

In one embodiment of the present disclosure, the at least one elastomeric film-forming polymer may be present in an amount ranging from 0.05% to 20% by weight, for example, from 0.1% to 15% by weight, or from 0.25% to 10% by weight, relative to the total weight of the composition.

The at least one elastomeric film-forming polymer may be chosen from homopolymers and copolymers. Non-limiting examples of elastomeric film-forming polymers include polyvinyl alcohols, polymers comprising at least one (meth)acrylic unit, and combinations thereof. In one embodiment, the at least one elastomeric film-forming polymer is a non-crosslinked polymer.

If necessary to modify the properties of the at least one elastomeric polymer, the cosmetic composition may further comprise at least one plasticizer and/or at least one auxiliary agent for facilitating the formation of an elastomeric polymer film on the keratin materials. Such an auxiliary film-forming agent may be chosen from compounds known to those skilled in the art as being capable of performing the desired function, for example, plasticizers and coalescence agents. The at least one elastomeric film-forming polymer, optionally combined with said at least one plasticizer and/or said at least one auxiliary agent for facilitating film formation, is capable of forming a film, after evaporation of the cosmetically acceptable medium. This evaporation may be carried out in the open air or by providing heat, for example using a dryer.

French Patent No. FR-A-2 782 917 describes various suitable plasticizers and/or agents for facilitating film formation on keratin materials. For example, this plasticizer and/or agent may be chosen from known plasticizers or coalescence agents such as:
glycols and their derivatives, such as diethylene glycol ethyl ether, diethylene glycol methyl ether, diethylene glycol butyl ether, diethylene glycol hexyl ether, ethylene glycol ethyl ether, ethylene glycol butyl ether, ethylene glycol hexyl ether, and pentylene glycol,
glyceryl esters,
propylene glycol derivatives, for example, propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether, and propylene glycol butyl ether,
acid esters, such as carboxylic acid esters, for example, citrates, phthalates, adipates, carbonates, tartrates, phosphates, and sebacates, and
mixtures thereof.

The amount of plasticizer and/or of film-forming agent may be chosen by those skilled in the art so as to obtain a polymeric system (elastomeric polymer+plasticizer and/or film-forming agent) that results in a film having the desired mechanical properties, while at the same time allowing the composition to conserve the desired cosmetic properties. This amount may range from 0.01% to 25% of the total weight of the composition, for example, from 0.01% to 15% of the total weight of the composition.

The composition according to the invention may also contain at least one cosmetically acceptable medium. As used herein, the term “cosmetically acceptable medium” is intended to mean a non-toxic medium that can be applied to human keratin materials such as the scalp and the hair and can be sold without a medical prescription.

In one embodiment, the at least one cosmetically acceptable medium of the composition may comprise a hydrophilic phase comprising water and/or at least one water-miscible cosmetically acceptable solvent, such as C₁-C₄ monoalcohols and polyols, for instance, glycerol, diglycerol, propylene glycol, diethylene glycol, sorbitol, and glycol ethers. In another embodiment, the cosmetically acceptable medium of the composition may comprise water or at least one cosmetically acceptable solvent such as alcohols or water-solvent mixtures, for example, where the at least one solvent is chosen from C₁-C₄ alcohols. Non-limiting examples of suitable alcohols include ethanol, isopropanol, and t-butanol. In at least one embodiment, ethanol may be used.

The at least one elastomeric film-forming polymer of the invention may be soluble or water-dispersible in an aqueous or aqueous-alcoholic medium. In one embodiment, the at least one elastomeric film-forming polymer is soluble at at least 10 g of active material in 90 g of aqueous or aqueous-alcoholic medium (containing 70% water and 30% alcohol), at ambient temperature and atmospheric pressure.

In another embodiment of the present disclosure, the film may exhibit reduced water sensitivity, for example, in an atmosphere with a relative humidity of 30% to 80%, i.e., the film keeps its elastomeric properties for several hours, such that it is flexible and non-brittle, and correctly follows the movements of the skin and/or of the scalp. For example, from 30% to 80% relative humidity, the elongation at break of the film obtained may not vary by more than 50% (±400%) and/or its instantaneous recovery may not vary by more than 25% (±18.75%). In other words, from 30% to 80% relative humidity, the elongation at break of the film obtained ranges from 400% to 1200% and/or its instantaneous recovery ranges from 57% to 93%.

In a further embodiment of the present disclosure, the at least one cosmetically acceptable medium of the composition may comprise an oily phase comprising at least one fatty substance that is liquid at ambient temperature (22° C.±2° C.) and atmospheric pressure and water-immiscible, i.e., an oil. These oils may be chosen from emollient oils, for example, synthetic oils, such as fatty acid esters and/or fatty alcohol esters; oils of mineral or plant origin; and silicone oils having a linear or cyclic structure that may or may not be modified. These oils may be volatile or non-volatile.

The at least one cosmetically acceptable medium may also comprise at least one surfactant chosen from non-ionic surfactants, anionic surfactants, cationic surfactants, amphoteric surfactants, and mixtures thereof. In one embodiment, the at least one surfactant may be chosen from non-ionic surfactants, amphoteric surfactants, and mixtures thereof.

Non-limiting examples of suitable anionic surfactants include salts, for example, alkali metal salts such as sodium salts, ammonium salts, amine salts, amino alcohol salts, and alkaline earth metal salts such as magnesium salts, of the following types: alkyl sulphates, alkyl ether sulphates, alkylamido ether sulphates, alkylaryl polyether sulphates, monoglyceride sulphates; alkylsulphonates, alkylamidesulphonates, alkylaryl-sulphonates, α-olefin sulphonates, paraffin sulphonates, alkylsulphosuccinates, alkyl ether sulphosuccinates, alkylamide sulphosuccinates, alkylsulphoacetates, acylsarcosinates, and acylglutamates. In one embodiment, the alkyl and acyl groups of all these compounds may comprise from 6 to 24 carbon atoms and the aryl groups may be chosen from phenyl groups, benzyl groups, and mixtures thereof.

Suitable anionic surfactants may also include, but are not limited to, anionic disurfactants, for example, C₆-C₂₄ alkyl monoesters of polyglycoside dicarboxylic acids such as alkyl glucoside citrates, polyalkyl glycoside tartrates and polyalkyl glycoside sulphosuccinates, alkylsulphosuccinamates, acylisethionates, and N-acyltaurates. In one embodiment, the alkyl and acyl group of these compounds may comprise from 12 to 20 carbon atoms, and mixtures thereof. Anionic surfactants that may be used herein may also be chosen from acyllactylates in which the acyl group contains from 8 to 20 carbon atoms. Further non-limiting examples of anionic surfactants include alkyl-D-galactosideuronic acids and the salts thereof, polyoxyalkylenated (C₆₋₂₄)alkyl ether carboxylic acids and salts thereof, polyoxyalkylenated (C₆₋₂₄)alkyl(C₆₋₂₄)aryl ether carboxylic acids and salts thereof, polyoxyalkylenated (C₆₋₂₄)alkylamido ether carboxylic acids and salts thereof, and mixtures thereof. The ether carboxylic acids may comprise, for example, from 2 to 50 ethylene oxide units.

In one embodiment of the present disclosure, the anionic surfactants are chosen from alkyl sulphates, alkyl ether sulphates, alkyl ether carboxylates, and mixtures thereof. These anionic surfactants may be in a form chosen from alkali metal salts, alkaline earth metal salts, ammonium salts, amine salts, and amino alcohol salts.

Non-limiting examples of suitable amphoteric surfactants include derivatives of secondary or tertiary aliphatic amines, in which the aliphatic group may be a linear or branched chain comprising from 8 to 22 carbon atoms and comprising at least one anionic group such as, for example, a carboxylate, sulphonate, sulphate, phosphate, or phosphonate group. The amphoteric surfactant may be chosen from, for example, (C₈₋₂₀)alkylbetaines, sulphobetaines, (C₈₋₂₀)alkylamido(C₆₋₈)alkylbetaines, (C₈₋₂₀)alkylamido(C₆₋₈)alkylsulphobetaines, and mixtures thereof.

Non-limiting examples of amine derivatives include, but are not limited to, products sold under the name Miranol®, as described in U.S. Pat. Nos. 2,528,378 and 2,781,354, and classified in the CTFA dictionary, 3rd edition, 1982, under the names amphocarboxyglycinate and amphocarboxypropionate, having the respective structures (1) and (2):
Rₐ—CONHCH₂CH₂—N(R_{b})(R_{c})(CH₂COO⁻) (1)
in which:
   Rₐ is chosen from alkyl groups derived from an acid Rₐ—COOH present in hydrolysed coconut oil, heptyl groups, nonyl groups, and undecyl groups,
   R_{b} is a beta-hydroxyethyl group, and
   R_{c} is a carboxymethyl group; and
      Rₐ′—CONHCH₂CH₂—N(B)(C) (2)
in which:
   B is —CH₂CH₂OX′,
   C is —(CH₂)_{z}—Y′, wherein z is equal to 1 or 2,
   X′ is chosen from —CH₂CH₂—COOH and hydrogen,
   Y′ is chosen from —COOH and —CH₂—CHOH—SO₃H,
   Rₐ′ is chosen from alkyl groups of an acid Rₐ′—COOH present in coconut oil or in hydrolysed linseed oil; alkyl groups, for example, a C₁₇ alkyl group and its iso form; and an unsaturated C₁₇ group.

The compounds of structures (1) and (2) may be classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid. For example, cocoamphodiacetate is sold by the company Rhodia under the trade name Miranol® C2M concentrate.

In one embodiment of the present disclosure, the amphoteric surfactant is chosen from (C₈₋₂₀)alkylbetaines, (C₈₋₂₀)alkylamido(C₆₋₈)alkylbetaines, alkylamphodiacetates, and mixtures thereof.

Non-limiting examples of suitable non-ionic surfactants include compounds described in particular in the book “Handbook of Surfactants” by M. R. Porter, published by Blackie & Son (Glasgow and London), 1991, pp. 116-178, such as alcohols; alpha-diols; (C₁₋₂₀)alkylphenols; and polyethoxylated, polypropoxylated, and polyglycerolated fatty acids having a fatty chain comprising, for example, from 8 to 18 carbon atoms, wherein the number of ethylene oxide or propylene oxide groups may range from 2 to 50 and the number of glycerol groups may range from 2 to 30; and mixtures thereof.

The non-ionic surfactants may also be chosen from condensates of ethylene oxide and of propylene oxide with fatty alcohols; polyethoxylated fatty amides, for example, amides comprising from 2 to 30 ethylene oxide units; polyglycerolated fatty amides comprising, on average, from 1 to 5 glycerol groups, for example, from 1.5 to 4 gyclerol groups; ethoxylated fatty acid esters of sorbitan comprising from 2 to 30 ethylene oxide units; fatty acid esters of sucrose; fatty acid esters of polyethylene glycol; (C₆₋₂₄)alkylpolyglycosides; N—(C₆₋₂₄)alkylglucamine derivatives; amine oxides such as (C₁₀₋₁₄)alkylamine oxides and N—(C₁₀₋₄)acylaminopropylmorpholine oxides; and mixtures thereof. In one embodiment, the nonionic surfactants are chosen from (C₆₋₂₄)alkylpolyglycosides.

The total amount of surfactant in the cosmetic composition may range from 0.01% to 50% by weight, for example, from 0.1% to 25% by weight, relative to the total weight of the composition.

In another embodiment of the present disclosure, the composition may also contain at least one additional ingredient conventionally used in the cosmetic field, such as formulation additives and cosmetic active ingredients that have a beneficial effect on keratin materials such as the skin and keratin fibers such as the hair.

Non-limiting examples of suitable formulation additives include aqueous phase thickeners; fatty phase thickeners; anionic, non-ionic, cationic and amphoteric, fixing or non-fixing polymers; waxes; gums; fillers; fragrances; preserving agents; sequestering agents; pH modifiers such as basic solutions, for example, solutions of sodium hydroxide, of potassium hydroxide and of ammonium hydroxide, aminomethylpropanols, and primary, secondary, and tertiary amines; buffer solutions, for example, sodium bicarbonate; acid solutions, for example, solutions of citric acid or hydrochloric acid; pearlescent agents; dyestuffs and dye precursors; opacifiers; and any other additives conventionally used in cosmetic compositions which are intended to be applied to keratin materials such as the skin, the hair, and/or the eyelashes.

Suitable aqueous phase thickeners may include, but are not limited to, polymers such as carboxylic polymers and copolymers (Carbopol®); cellulose derivatives, such as carboxymethylcellulose, hydroxypropylcellulose, and hydroxyethylcellulose; guar gum and hydroxypropylated guar gums; electrolytes such as sodium chloride; hydrotopes such as sodium toluenesulphonate, potassium toluenesulphonate, and sodium xylenesulphonate; and mixtures thereof.

The composition of the invention may also contain at least one cosmetic active agent that has a beneficial effect on keratin materials, such as the zinc salts of organic acids (for example, acetates, glycolates, lactates, gluconates, and citrates) and inorganic acids (chlorides and sulphates); sunscreens; proteins; vitamins (for example, vitamins A, E, B₂, B₅, F, and C); provitamins; ceramides; pseudoceramides; free-radical scavengers; and plant extracts.

The amounts of the various optional additional ingredients in the compositions may be those generally used in the art, and may range, for example, from 0.001% to 20% of the total weight of the composition.

The compositions disclosed herein may be prepared according to the usual methods known in the art. The compositions can be packaged in various forms, for example, in bottles, optionally provided with a pump, heating bags, tubes, and simple pots for taking up the composition with the hands. The compositions may be in a form chosen from creams, gels, water-in-oil emulsions, oil-in-water emulsions, lotions, sprays, mousses, and waxes.

In one embodiment, the pH of the aqueous phase of the composition may range from 2 to 11, for example, from 3 to 10, or from 5 to 8. In another embodiment, the composition may have a dynamic viscosity, measured at ambient temperature and atmospheric pressure, of less than 200 cps, for example, ranging from 10 cps to 180 cps. This viscosity can be measured by any method known in the art, for example, using a Rheomat 180 at 25° C. and with a shear rate of 1 s⁻¹.

The compositions disclosed herein may be applied to keratin materials such as the hair, the skin, and the eyelashes, in a dry or wet state.

Of course, those skilled in the art will take care to choose the optional compound(s) to be added to the composition according to the invention in such a way that the advantageous properties intrinsically associated with the composition in accordance with the invention are not, or are not substantially, impaired by the envisaged addition.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

### Example 1

### Styling Mousse

The following composition was prepared by mixing the constituents listed below under “cold” conditions (i.e., ambient temperature) in a pressurized can.

| | | |
|---|---|---|
| Polyurethane (NMDEA¹⁾/PTMO 2900²⁾/IPDI³⁾ − 3/1/4) | 2% | AM⁴⁾ |
| Oxyethylenated sorbitan monolaurate | 0.2% | AM |
| Water | 92.8% | AM |
| Isobutane/propane (85/15) | 5% | AM |

¹⁾N-methyldiethanolamine
²⁾Poly(tetramethylene oxide) having a weight-average mass of 2900
³⁾Isophorone diisocyanate
⁴⁾Percent by mass active material

The polyurethane copolymer was prepared as described in French Patent No. FR-A-2 815 350, for example, according to the methods for synthesizing the polyurethane PU2 of Example 1, replacing the poly(tetramethylene oxide) having a weight-average molecular mass of 1400 with a poly(tetramethylene oxide) having a weight-average molecular mass of 2900.

The polyurethane copolymer formed had the following characteristics, measured as described above:
ε_{b}=1500%
Rᵢ=82%
R₃₀₀=92%
water-soluble at at least 10 g per liter.

This composition can be applied to dry or wet hair. It conferred good styling properties on the hairstyle (good degree of setting and very good hold of the hairstyle over time), that followed the movements of the head of hair.

### Example 2

### Styling Gel

The following composition was prepared by mixing the constituents listed below under “cold” condiitons.

| | | |
|---|---|---|
| Polyurethane (NMDEA/PTMO 2900/IPDI − 3/1/4) | 4% | AM |
| Jaguar HP 105 (Rhodia) | 1% | AM |
| Water | qs 100% | AM |

This styling gel gave the hair good styling properties (good degree of setting and very good hold of the hairstyle over time, flexible), without any helmet effect.

What is claimed is:

1. A leave-in cosmetic composition comprising, in a cosmetically acceptable medium, at least one elastomeric film-forming polymer, wherein the film obtained by drying said polymer at ambient temperature and a relative humidity of 55%±5%, has a mechanical profile comprising:
(a) an elongation at break (ε_{b}) of greater than or equal to 800%,
(b) an instantaneous recovery (Rᵢ) of at least 75%, after an elongation of 150%, and
(c) a recovery (R₃₀₀) at 300 seconds of greater than 80%.

2. The composition of claim 1, wherein the elastomeric film-forming polymer is soluble in a medium chosen from aqueous and aqueous-alcoholic mediums.

3. The composition of claim 1, wherein at from 30% to 80% relative humidity, the elongation at break of the film ranges from 400% and 1200% and/or the instantaneous recovery of the film ranges from 57% and 93%.

4. The composition of claim 1, further comprising at least one surfactant.

5. The composition of claim 4, wherein the at least one surfactant is present in an amount ranging from 0.01% to 50% by weight, relative to the total weight of the composition.

6. The composition of claim 5, wherein the at least one surfactant is present in an amount ranging from 0.1% to 25% by weight, relative to the total weight of the composition.

7. The composition of claim 4, wherein the at least one surfactant is chosen from non-ionic surfactants, amphoteric surfactants, and mixtures thereof.

8. The composition of claim 1, wherein said composition is in a form chosen from mousses and gels for styling the hair.

9. The composition of claim 6, wherein the composition has a dynamic viscosity, measured at ambient temperature and atmospheric pressure, of less than 200 cps.

10. The composition of claim 7, wherein the composition has a dynamic viscosity, measured at ambient temperature and atmospheric pressure, of less than 200 cps.

11. The composition of claim 1, wherein the at least one elastomeric film-forming polymer is present in an amount ranging from 0.05% to 20% by weight, relative to the total weight of the composition.

12. The composition of claim 11, wherein the at least one elastomeric film-forming polymer is present in an amount ranging from 0.1% to 15% by weight, relative to the total weight of the composition.

13. The composition of claim 11, wherein the at least one elastomeric film-forming polymer is present in an amount ranging from 0.25% to 10% by weight, relative to the total weight of the composition

14. The composition of claim 1, wherein the at least one elastomeric film-forming polymer is chosen from polyurethanes, polyvinyl alcohols, polymers comprising at least one (meth)acrylic unit, and combinations thereof.

15. The composition of claim 1, wherein the cosmetically acceptable medium comprises a hydrophilic phase containing water and/or at least one water-miscible cosmetically acceptable solvent.

16. The composition of claim 15, wherein the at least one solvent is chosen from C₁-C₄ alcohols.

17. The composition of claim 1, further comprising at least one additional ingredient chosen from cosmetic active ingredients and formulation additives.

18. A method for conditioning human keratin materials, comprising applying a leave-in cosmetic composition to the human keratin materials,
wherein the leave-in cosmetic composition comprises, in a cosmetically acceptable medium, at least one elastomeric film-forming polymer; and
wherein the film obtained by drying this polymer at ambient temperature and a relative humidity of 55%±5%, has a mechanical profile comprising:
(a) an elongation at break (ε_{b}) of greater than or equal to 800%,
(b) an instantaneous recovery (Rᵢ) of at least 75%, after an elongation of 150%, and
(c) a recovery (R₃₀₀) at 300 seconds of greater than 80%.

19. A method for caring for and/or protecting the skin and/or human keratin fibers, comprising applying a leave-in cosmetic composition to the skin and/or the human keratin fibers,
wherein the leave-in cosmetic composition comprises, in a cosmetically acceptable medium, at least one elastomeric film-forming polymer; and
wherein the film obtained by drying this polymer at ambient temperature and a relative humidity of 55%±5%, has a mechanical profile comprising:
(a) an elongation at break (ε_{b}) of greater than or equal to 800%,
(b) an instantaneous recovery (Rᵢ) of at least 75%, after an elongation of 150%, and
(c) a recovery (R₃₀₀) at 300 seconds of greater than 80%.

20. A method for shaping human hair into a hairstyle and/or maintaining the hairstyle, comprising applying a leave-in cosmetic composition to the human hair,
wherein the leave-in cosmetic composition comprises, in a cosmetically acceptable medium, at least one elastomeric film-forming polymer, and
wherein the film obtained by drying this polymer at ambient temperature and a relative humidity of 55%±5%, has a mechanical profile comprising:
(a) an elongation at break (ε_{b}) of greater than or equal to 800%,
(b) an instantaneous recovery (Rᵢ) of at least 75%, after an elongation of 150%, and
(c) a recovery (R₃₀₀) at 300 seconds of greater than 80%.

21. A composition for the care and/or protection of keratin materials, comprising a leave-in cosmetic composition comprising, in a cosmetically acceptable medium, at least one elastomeric film-forming polymer, wherein the film obtained by drying said polymer at ambient temperature and a relative humidity of 55%±5%, has a mechanical profile comprising:
(a) an elongation at break (ε_{b}) of greater than or equal to 800%,
(b) an instantaneous recovery (Rᵢ) of at least 75%, after an elongation of 150%, and
(c) a recovery (R₃₀₀) at 300 seconds of greater than 80%.

22. The composition of claim 21, wherein the keratin materials are chosen from the skin, the hair, the eyelashes, and the nails.

23. A cosmetic hair product for maintaining and/or setting the hairstyle, comprising a leave-in cosmetic composition comprising, in a cosmetically acceptable medium, at least one elastomeric film-forming polymer, wherein the film obtained by drying said polymer at ambient temperature and a relative humidity of 55%±5%, has a mechanical profile comprising:
(a) an elongation at break (ε_{b}) of greater than or equal to 800%,
(b) an instantaneous recovery (Rᵢ) of at least 75%, after an elongation of 150%, and
(c) a recovery (R₃₀₀) at 300 seconds of greater than 80%.
